(19) United States
(12) Patent Application Publication
(10) Pub. No.: US 2008/0051671 A1
(43) Pub. Date: Feb. 28, 2008

(54) INTRAVASCULAR FILTER MONITORING

(75) Inventors: Thomas E. Broome, Shakopee, MN (US); Anthony C. Vrba, Maple Grove, MN (US); Narin Anderson, Savage, MN (US); Justin Crank, St. Louis Park, MN (US); James G. Hansen, Coon Rapids, MN (US); Horng-Ban Lin, Maple Grove, MN (US); Mark Smith, Coon Rapids, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/929,007

(22) Filed: Oct. 30, 2007

Related U.S. Application Data
(63) Continuation of application No. 10/306,288, filed on Nov. 27, 2002.

(51) Int. Cl. A61B 5/026

(52) U.S. Cl. 600/504

(57) ABSTRACT

Devices and methods for monitoring the flow of blood through an intravascular device are disclosed. An apparatus for monitoring blood flow in accordance with an exemplary embodiment of the present invention includes an intravascular device coupled to an elongated member, a first sensor adapted to measure fluidic pressure proximal the intravascular device, a second sensor adapted to measure fluidic pressure distal the intravascular device, and a control unit for comparing the signals received from the first and second sensors to determine the pressure drop across the intravascular device.

### RELATED APPLICATIONS

This application is a continuation application of U.S. application Ser. No. 10/306,288 filed Nov. 27, 2002.

### FIELD OF THE INVENTION

The present invention relates generally to the field of intravascular filter monitoring. More specifically, the present invention pertains to devices and methods for monitoring the flow of blood through an embolic protection filter.

### BACKGROUND OF THE INVENTION

Intravascular devices such as embolic protection filters are generally placed with the lumen of a blood vessel or artery to filter embolic debris dislodged during a therapeutic procedure such as percutaneous transluminal coronary angioplasty (PTCA), percutaneous extraction atherectomy, or stent delivery. To filter the dislodged embolic debris, an embolic protection filter can be placed distally of the therapeutic device (e.g. an angioplasty or atherectomy catheter) and deployed within the patient's vessel or artery. Over time, the embolic protection filter may become occluded with the embolic debris, necessitating the removal and/or replacement of the filter from the vessel.

Although many techniques have been developed to monitor the flow of blood through a patient's body, real-time monitoring of blood flow through an embolic protection filter can often prove difficult. For example, in a fluoroscopic monitoring technique, a contrast material is periodically injected into a vein or artery at pre-determined intervals throughout the course of a therapeutic procedure. The contrast media, which is visible under a fluoroscopic monitor, can be utilized to monitor the flow of blood through the vasculature, to determine the patency of a specific artery or vessel, and to assess the severity of the lesion or stenosis.

One particular issue associated with fluoroscopic monitoring, however, is the ability to readily monitor the flow of blood through an embolic protection filter. Since fluoroscopic monitoring may require as much as several minutes to perform, such techniques are not well suited for real-time monitoring of blood flow through an embolic protection filter.

### SUMMARY OF THE INVENTION

The present invention relates generally to the field of intravascular filter monitoring. In an exemplary embodiment, an apparatus for monitoring blood flow across an intravascular device comprises an elongated member having a proximal end and a distal end, an intravascular device disposed about the elongated member proximal the distal end thereof, a first sensor adapted to measure blood flow or pressure proximal the intravascular device, and a second sensor adapted to measure blood flow or pressure distal the intravascular device. A control unit located outside of the patient's body may be used to determine the pressure drop across the intravascular device.

### BRIEF DESCRIPTION OF THE DRAWINGS

FIG. 1 is a plan view of an apparatus for measuring blood flow through an embolic protection filter in accordance with an exemplary embodiment of the present invention;

FIG. 2 is a cross-sectional view of the apparatus of FIG. 1, showing the first sensor located proximal the embolic protection filter; and

FIG. 3 is a cross-sectional view of the apparatus of FIG. 1, showing the second sensor located distal the embolic protection filter; and

FIG. 4 is a plan view of an apparatus for measuring blood flow across an angioplasty balloon in accordance with another exemplary embodiment of the present invention.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, materials and manufacturing processes are illustrated for the various elements, those skilled in the art will recognize that many of the examples provided have suitable alternatives that may be utilized.

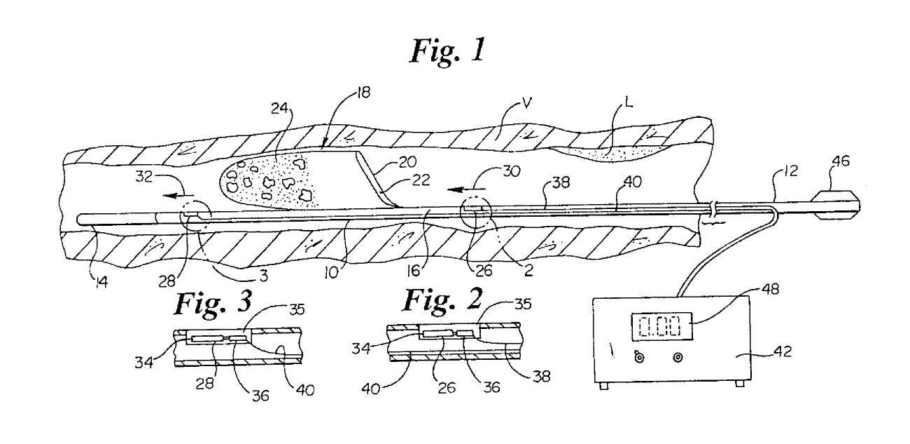

FIG. 1 is a plan view of an apparatus for monitoring the flow of blood through an intravascular device in accordance with an exemplary embodiment of the present invention. As shown in FIG. 1, an elongated member 10 is inserted into a patient's vessel V at least in part distal a lesion L. Elongated member 10 may be a tubular member having a proximal end 12, a distal end 14, and an inner lumen 16. An optional hub 46 attached to the proximal end 12 of elongated member 10 can be utilized to facilitate advancement of the device through the patient's vasculature.

In certain embodiments of the present invention, elongated member 10 may comprise a guidewire or filterwire adapted to permit an intravascular device such as an angioplasty catheter or embolic protection filter to slide thereon. In other implementations, elongated member 10 may form part of a catheter that can be advanced along a separate wire disposed within the patient's vasculature. For example, elongated member 10 may form part of an angioplasty catheter having an angioplasty balloon adapted to perform a therapeutic procedure such as percutaneous transluminal coronary angioplasty (PTCA).

In the exemplary embodiment shown in FIG. 1, elongated member 10 is formed from, for example, a hypo-tube or a polymeric material. Examples of suitable polymeric materials include polypropylene (PP), polyvinylchloride (PVC), polytetrafluoroethylene (PTFE), and polyether block amide (PEBA). Polyether block amide is commercially available from Atochem Polymers of Birdsboro, Pa. under the trade name PEBAX.

Elongated member 10 may also include a polymeric coating to facilitate advancement through the tortuous vasculature, and to reduce tissue damage in the patient. Examples of suitable polymeric coatings include polyacrylic acid, polycaprolactone, polycarboxylic acid, polyamide, polyvinyl ether, polyurethane, polytetrafluoroethylene, and polyorthoesters. Polyacrylic acid is commercially available from Boston Scientific Corporation of Natick, Mass. under the trade name HYDROPASS.

Attached to a distal portion of elongated member 10 is an embolic protection filter 18. One type of embolic protection filter 18 includes a support hoop 20 forming a mouth or opening 22 for collecting embolie debris. As shown in FIG. 1, the support hoop 20 can be configured to support the embolic protection filter 18 within vessel V. In some embodiments, the support hoop can be configured to provide full 360° wall apposition of the embolic protection filter 18 within vessel V, if desired.

A filter membrane 24 attached to the support hoop 20 is adapted to filter embolic debris contained within vessel V. Filter membrane 24 may comprise a braided wire mesh formed of a metallic material such as stainless steel, platinum, or nickel-titanium alloy (Nitinol). Alternatively, filter membrane 24 may comprise a microporous membrane made from a polymeric material such as polypropylene (PP), polyurethane, polyethylene terapthlalate, polyether-ether ketone (PEEK), polyether block amide (PEBA), polyamide (nylon), polyvinylchloride (PVC), polytetrafluoroethylene (PTFE) or any mixture, blend or combination thereof.

Elongated member 10 further includes a first sensor 26 coupled to the elongated member 10 proximal the embolic protection filter 18, and a second sensor 28 coupled to the elongated member 10 distal the embolic protection filter 18. The first and second sensors 26, 28 are configured to respond to changes in blood flow or pressure at locations 30 and 32 within vessel V, and output a corresponding electrical signal to a control unit 42 located outside the patient's body.

The first and second sensors 26, 28 each include a transducer capable of producing an electrical signal in response to fluidic pressure within vessel V. As shown in greater detail in FIGS. 2-3, each transducer 34 may comprise a strain gauge mounted at least in part within a groove 35 formed on the outer surface of the elongated member 10. Examples of strain gauges suitable for use with the present invention include capacitive, resistive, inductive, or piezoelectric-type strain gauges.

A metallic bonding pad 36 may be used to connect each transducer element 34 to a set of leads 38, 40 disposed in part within the inner lumen 16 of elongated member 10.

Connection of the leads 38, 40 to the bonding pads 36 may be accomplished by any suitable attachment mechanism, including soldering, welding or crimping. As shown in FIG. 1, the leads 38, 40 extend proximally through inner lumen 16, and exit at a port 44 located at or near the proximal end 12 of the elongated member 10. An optional protective sleeve or coating may be applied to each set of leads 38, 40 to provide a layer of insulation, if desired.

In another exemplary embodiment in accordance with the present invention, the first and second sensors 26, 28 may comprise ultrasonic transducers adapted to measure the flow of blood using ultrasonic waves or pulses. A first ultrasonic transceiver is operatively coupled to the outside of elongated member 10 proximal the embolic protection filter 18. A second ultrasonic transceiver is operatively coupled to the elongated member 10 distal the embolic protection filter 18. As with the previous embodiment, several leads 38, 40 may be used to connect the first and second ultrasonic sensors to the control unit 42 located outside the patient's body.

In use, the first and second ultrasonic transceivers transmit an ultrasonic wave or pulse that can be subsequently received. As the wave travels from the source to the receiver, the velocity of the wave will either increase or decrease due to the Doppler effect resulting from the flow of blood through the vessel V. The velocity of the blood can then be determined by measuring the difference in travel time or the relative phase shift between the source (i.e. upstream) wave and the received (i.e. downstream) wave. As with any of the other techniques described herein, the pressure drop through the embolic protection filter can then be determined by comparing (i.e. subtracting) the respective values obtained from both the first and second transceivers to obtain a differential value representing the pressure drop through the embolic protection filter 18.

In yet another exemplary embodiment in accordance with the present invention, the first and second sensors 26, 28 may comprise microelectrical mechanical system (MEMS) sensors. Each MEMS sensor 26, 28 may be embedded at least in part within a grove 35 formed on the outer surface of the elongated member 10. An optional primer coating may be applied to the groove 35 to facilitate attachment of the MEMS sensor therein. If desired, a second coating (e.g. polyimide or silicon rubber) may also be applied to each sensor to insulate the sensor once placed within the groove.

In certain embodiments, the electrical signal outputted from each MEMS sensor may be transmitted through several leads operatively connected to a control unit located outside the patient's body. In other embodiments, the electrical signal outputted from each MEMS sensor may be wirelessly transmitted to an antennae located outside of the patient's body. In either embodiment, the control unit 42 is configured to receive the electrical signals from each MEMS sensor, and determine the pressure drop through the embolic protection filter 18.

While the exemplary embodiment of FIG. 1 illustrates an apparatus having sensors coupled directly to the elongated member 10, other embodiments have been envisioned in which one or more sensors placed outside of the patient's body may be used to measure the pressure drop through the embolic protection filter. For example, an apparatus for monitoring the flow of blood through an embolic protection filter may include an elongated tubular member having a first opening located proximal the embolic protection filter, and a second opening located distal the embolic protection filter. The first opening is configured to transmit blood through a first lumen to a first sensor located outside the patient's body. The second opening is configured to transmit blood through a second lumen to a second sensor located outside the body. In use, the first and second sensors, which are in fluid communication with the first and second openings, can be utilized to obtain a measure of the blood flow or pressure both proximal and distal the embolic protection filter.

To determine the pressure drop through the embolic protection filter 18, a control unit 42 may be used with any of the embodiments discussed herein. Control unit 42 includes a comparator circuit configured to take an electrical signal received from the first sensor 26, and compare that signal to an electrical signal received from the second sensor 28 to determine a differential value. From this differential value, a measure of the pressure drop through the embolic protection filter 18 can be obtained and outputted to a screen 48 located on the control unit 42.

Control unit 42 may further include a calibration device to calibrate the first and second sensors 26, 28, and reset the calibration device to zero-out the control unit 42 prior to the collection of embolic debris within the embolic protection filter 18. The calibration device can be utilized to selectively change the sensitivity of the first and/or second sensors 26, 28, and to compensate for environmental variables such as the size of the vessel, the location or position of the device within the vasculature, and the type of intravascular device employed. For example, if a resistive-type strain gauge is used, the calibration device can include a Wheatstone bridge circuit to balance the resistance of the gauge.

Control unit 42 may further optionally include a signaling device to notify the physician when the pressure drop within the embolic protection filter 18 has reached a pre-determined value. For example, control unit 42 may include an audible signal configured to sound when the pressure drop through the filter reaches a certain threshold value pre-determined by the operator. Control unit 42 may also include an LED or other visual indicator that can be actuated when the pressure drop through the embolic protection filter reaches a certain level.

A method in accordance with the present invention includes the steps of transluminally inserting the elongated member 10 into a vessel V and advancing the device to a desired location distal a lesion L. Once the elongated member 10 is in place, the embolic protection filter 18 can then be deployed within the vessel, as shown in FIG. 1. With the embolic protection filter 18 deployed in vessel V, the physician can then calibrate the device by obtaining an initial (i.e. calibration) reading from each of the sensors 26, 28, and then comparing the difference to obtain an initial differential value. If desired, the control unit 42 can then be set to zero prior to collecting embolic debris within the embolic protection filter 18.

To monitor the flow of blood through the embolic protection filter 18, control unit 42 continuously and repeatedly receives and compares the signals received from the first and second sensors 26, 28 to obtain a differential value. This differential value is outputted to a screen 48 located on the control unit 42. As the embolic protection filter 18 becomes occluded with embolic debris dislodged during the therapeutic procedure, the flow of blood at second location 32 decreases in comparison to the flow of blood at first location 30. When the differential value measured by the control unit 42 reaches a certain threshold level, the signaling device can be actuated to notify the physician that the embolic protection filter l 8 may need to be removed and/or replaced.

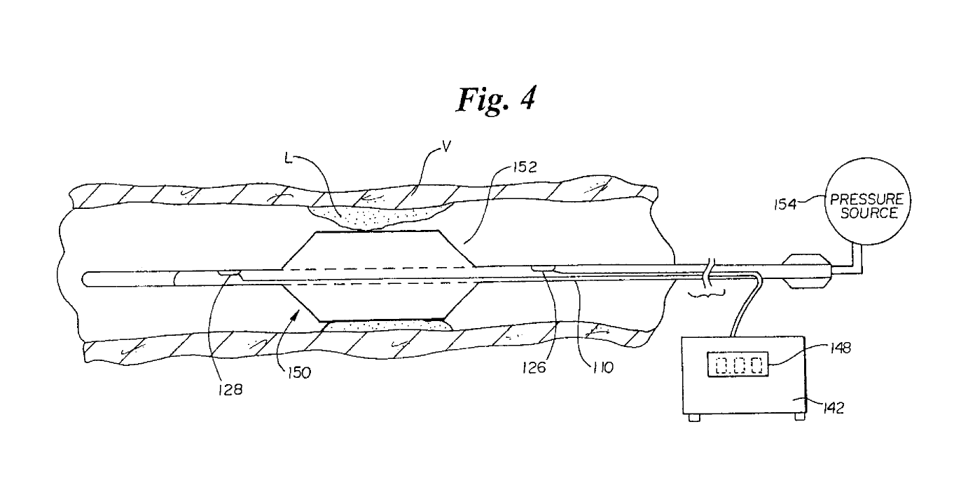

Although the exemplary embodiment described with respect to FIG. 1 illustrates determining the pressure drop across an embolic protection filter, it is to be understood that other intravascular devices can be measured with the apparatus and methods described herein. In one embodiment illustrated in FIG. 4, for example, the elongated tubular member 110 may form part of an angioplasty catheter 150 having a angioplasty balloon 152 that can be expanded within vessel V. Similar to the embodiment illustrated in FIG. 1, a first sensor 126 can be coupled to the elongated member 110 proximal the balloon 152, and a second sensor 128 can be coupled to the elongated member 110 distal the balloon 152. The angioplasty balloon 152 is in fluid communication with an external fluid source 154, and can be inflated between a collapsed position and an expanded position within vessel V.

In use, the elongated member 110 can be inserted transluminally into a vessel and advanced to the site of the lesion L to perform an angioplasty procedure such as percutaneous transluminal coronary angioplasty (PTCA). Once positioned, the operator next calibrates the device while the balloon 52 is in the collapsed (i.e. unexpanded) position to obtain an initial reading from each of the sensors 126, 128. A control unit 142 similar to that described with respect to FIG. 1 can be utilized to calibrate the sensors 126, 128, if necessary.

Once the operator has positioned the apparatus adjacent the lesion L, and has obtained an initial (i.e. calibration) reading from each of the sensors 126, 128, the balloon 152 is then inflated within vessel V, forcing the lesion L to become dislodged from the vessel wall. As the balloon 152 is inflated, the pressure differential measured by the first and second sensors 126, 128 increases as a result of the occlusion within vessel V created by the balloon 152. This increase in pressure differential can be outputted to the screen 148 on the control unit 142 to provide the operator with feedback that the balloon 152 has been engaged within the vessel V. An alarm can be activated when the pressure differential has reached a certain pre-determined level, or when the second pressure sensor 128 measures a no-flow condition, indicating total occlusion within the vessel V.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. An apparatus for monitoring blood flow past an inflatable balloon, the apparatus comprising:
an angioplasty catheter having a proximal end and a distal end;
an angioplasty balloon coupled to the angioplasty catheter proximal of the distal end thereof;
a first sensor adapted to measure a blood flow characteristic coupled to the angioplasty catheter proximal to the angioplasty balloon; and
a second sensor adapted to measure a blood flow characteristic coupled to the angioplasty catheter distal to the angioplasty balloon.

2. The apparatus of claim 1, wherein the blood flow characteristic comprises blood flow rate.

3. The apparatus of claim 1, wherein the blood flow characteristic comprises blood pressure.

4. The apparatus of claim 1, further comprising a hub attached to the proximal end of the angioplasty catheter.

5. The apparatus of claim 1, wherein the first and second sensors comprise strain gauges.

6. The apparatus of claim 5, wherein the strain gauges are selected from the group consisting of resistive, capacitive, inductive and piezoelectric-type strain gauges.

7. The apparatus of claim 1, wherein the first and second sensors comprise ultrasonic sensors.

8. The apparatus of claim 1, wherein the first and second sensors comprise MEMS sensors.

9. The apparatus of claim 8, wherein the MEMS sensors comprise wireless MEMS sensors.

10. The apparatus of claim 1, further comprising a control unit for monitoring the signals received from the first and second sensors, said control unit comprising a comparator circuit for determining a pressure drop past the angioplasty balloon.

11. The apparatus of claim 10, wherein the control unit comprises calibration means for calibrating said first and second sensors, and reset means for resetting the control unit.

12. The apparatus of claim 11, wherein said control unit includes alarm means to notify the operator when the pressure drop past the angioplasty balloon has reached a pre-determined value.

13. The apparatus of claim 1, wherein the angioplasty balloon is in fluid communication with an external fluid source that can be used to inflate the angioplasty balloon from a collapsed position to an expanded position.

14. A method of monitoring blood flow past a lesion, the method comprising the steps of:
providing an apparatus comprising:
an angioplasty catheter having a proximal end and a distal end;
an angioplasty balloon coupled to the angioplasty catheter proximal of the distal end thereof;
a first sensor adapted to measure a blood flow characteristic coupled to the angioplasty catheter proximal to the angioplasty balloon;
a second sensor adapted to measure a blood flow characteristic coupled to the angioplasty catheter distal to the angioplasty balloon; and
a control unit;
advancing the apparatus through a body lumen such that the angioplasty balloon is proximate a lesion;
inflating the angioplasty balloon to dislodge the lesion; and
monitoring a pressure drop across the angioplasty balloon.

15. The method of claim 14, wherein the control unit includes calibration means to calibrate the first and second sensors, and the method further comprises the step of calibrating the first and second sensors subsequent to the step of advancing the apparatus through a body lumen.

16. The method of claim 14, wherein the control unit includes alarm means to notify the operator when the pressure drop through the embolic protection filter has reached a pre-determined value, and the method further comprises the step of activating said alarm means when said pressure drop reaches the pre-determined level.

17. The method of claim 14, wherein the control unit includes alarm means to notify the operator when the distal sensor registers a no-flow condition, and the method further comprises the step of activating said alarm means when the distal sensor registers a no-flow condition.
